(19) United States
(12) Patent Application Publication
(10) Pub. No.: US 2008/0119548 A1
(43) Pub. Date: May 22, 2008

(54) Control Of Feeding Behavior By Changing Neuronal Energy Balance

(76) Inventors: Gabrielle V. Ronnett, Baltimore, MD (US); Frankcis P. Kuhajda, Baltimore, MD (US); Jagan N. Thupari, Baltimore, MD (US); Leslie E. Landree, Baltimore, MD (US); Timothy H. Moran, Baltimore, MD (US); Eun-Kyoung Kim, Baltimore, MS (US)

Correspondence Address: JACOBSON HOLMAN PLLC, 400 SEVENTH STREET N.W., SUITE 600, WASHINGTON, DC, 20004, US

(21) Appl. No.: 10/593,090

(22) PCT Filed: Mar. 18, 2005
(86) PCT No.: PCT/US05/09069

§ 371 Date: Oct. 26, 2007

Related U.S. Application Data
(60) Provisional application No. 60/554,228, filed on Mar. 18, 2004.

(51) Int. Cl. A61K 31/341; A61P 3/04

(52) U.S. Cl. 514/473

(57) ABSTRACT

Obesity is a worldwide health issue, affecting children and adults in developed and developing countries. Obesity is a disorder of both energy metabolism and appetite regulation, and may be understood as a dysfunction of energy balance. Applicants have found a means for regulating food intake by a subject by administering a compound to the subject which affects neuronal energy balance. Applicants have found a means for regulating food intake by a subject administering a compound to the subject which targets the activity of AMPK, in particular inhibiting activation, in particular hypothalamic. Applicants have also found a method of inducing weight loss in a subject by decreasing the subjects appetite by administering a compound to the subject which increases the subject's neuronal energy balance.

### BACKGROUND

Obesity is a worldwide health issue, affecting children and adults in developed and developing countries. Obesity is a disorder of both energy metabolism and appetite regulation, and may be understood as a dysfunction of energy balance.

Despite significant advances in the understanding of appetite and satiety at molecular levels, practical therapies for weight loss remain elusive. C75, a synthetic fatty acid synthase (FAS) inhibitor identified in U.S. Pat. No. 5,981,575 (incorporated herein by reference), causes profound weight loss and anorexia in lean, diet-induced obese (DIO), and genetically obese (ob/ob) mice. International Patent Application PCT/US03/03839 describes that, in addition to FAS inhibition, C75 also stimulates carnitine palmitoyltransferase-1 (CPT-1) activity, increasing fatty acid oxidation and ATP levels. As described by Kim, et al., enzymes of the fatty acid metabolic pathways are highly expressed in hypothalamic neurons that regulate feeding behavior (Am J Physiol Endocrinol Metab 283, E867-79 (2002).). Therefore, alterations in fatty acid metabolism may affect neuronal energy flux, which could signal a change in energy status, leading to changes in feeding behavior.

AMPK (AMP-activated protein kinase) is activated by metabolic stresses such as nutrient starvation and ischemia-hypoxia and by physiological processes such as vigorous exercise. Increases in the AMP/ATP ratio, decreases in cellular pH, and increases in the creatine/phosphocreatine ratio are known to activate AMPK via allosteric activation of AMPK by AMP and phosphorylation of AMPK by AMPKK.

Once activated, AMPK switches off ATP-consuming biosynthetic pathways such as fatty acid synthesis, and switches on ATP-generating metabolic pathways such as fatty acid oxidation to preserve ATP levels. The central roles of AMPK in both energy sensing and the control of fatty acid metabolism and its regulation by leptin in muscle make it a candidate metabolic sensor in the hypothalamus to relay changes in metabolism caused by C75 and other compounds.

### SUMMARY OF THE INVENTION

Applicants have found a means for regulating food intake by a subject by administering a compound to the subject which affects neuronal energy balance.

Applicants have found a means for regulating food intake by a subject administering a compound to the subject which targets the activity of AMPK, in particular inhibiting AMPK activation, in particular hypothalamic AMPK.

Applicants have also found a method of inducing weight loss in a subject by decreasing the subject's appetite by administering a compound to the subject which increases the subject's neuronal energy balance.

### DESCRIPTION OF THE FIGURES

FIG. 1. Food intake is affected by C75, AICAR or compound C.

(a) BALB/c male mice (n=7-9) received an i.c.v. injection of either 2.5 μl of RPMI with or without C75 (5 or 10 μg), and food intake was monitored as described in Methods.

(b) Mice (n=4-10) received an i.c.v. injection of 2.5 μl of saline with or without AICAR (1 or 3 μg), and food intake was monitored.

(c) Food intake was measured from mice (n=7-8) received an i.c.v. injection of 2.5 μl of saline with or without compound C (2 or 5 μg).

(d) Changes in bodyweight 24 hr after i.c.v. injection of C75, AICAR or compound C (n=4-10).

(e) Two hundred μl of vehicle (saline) or saline containing compound C (10 or 30 mg/kg bodyweight) was administered i.p. to mice (n=4-7).

(f) Two hundred μl of vehicle (RPMI) or RPMI containing C75 (10 mg/kg bodyweight) was administered i.p. to mice (n=4-9). Data were combined from three experiments. *, p<0.05; **, p<0.01; ***, p<0.001, compared to vehicle RPMI or saline treatment.

FIG. 2. C75 treatment reduces the phosphorylation of hypothalamic AMPKα.

(a) Levels of phosphorylated AMPKα (α1 and α2) and total AMPKα (α1 and α2) were visualized by Western blot analysis in extracts of hypothalamus at various times after i.c.v. injection of C75 (5 or 10 mg) at onset of dark cycle.

(c,e) Levels of phosphorylated AMPKα and total AMPKα from hypothalamus (c) or liver (e) after i.p. injection of C75 (10 mg/kg bodyweight). Tissue samples were prepared 1 hr after the i.p. injection. (c and e). Quantification from Western blot (FIGS. 2a,c and e) represents the fold-difference in ratio (phosphorylated AMPKα/total AMPKα) compared to control. The sensitivity of signal detection for phosphorylated AMPKα is 100-fold higher than total AMPKα.

FIG. 3. C75 also reduces the fasting-induced phosphorylation of hypothalamic AMPKα.

(a) Levels of phosphorylated AMPKα and total AMPKα were visualized in the hypothalamus from control (ad libitum access to food) and fasted mice. Food was withdrawn at onset of dark cycle (0 hr) over 24 hr, and the levels of phosphorylated and total AMPKα was determined at 0 hr, 3 hr and 24 hr after fasting.

(b) The graphs show the fold-difference from quantification of Western blot (FIG. 3a).

(c) Levels of phosphorylated and total hypothalamic AMPKα were determined in mice that were fasted for 24 hr and then received an i.p. injection of RPMI with or without C75 (10 mg/kg bodyweight). Tissue samples were prepared 1 hr after the i.p. injection.

(d) The graphs show the fold-difference from quantification of Western blot (FIG. 3c).

FIG. 4. C75 alters ATP level of hypothalamic neuron and AICAR reverses both C75-induced anorexia and reduction in pAMPKα levels.

(a) Primary hypothalamic neurons were treated with 20 or 40 mg/ml of C75 for 30 min and 2 hr. ATP levels were evaluated by luminescence and represented as a % of untreated controls (−). Data were combined from three independent experiments. **, p<0.01 compared to untreated control.

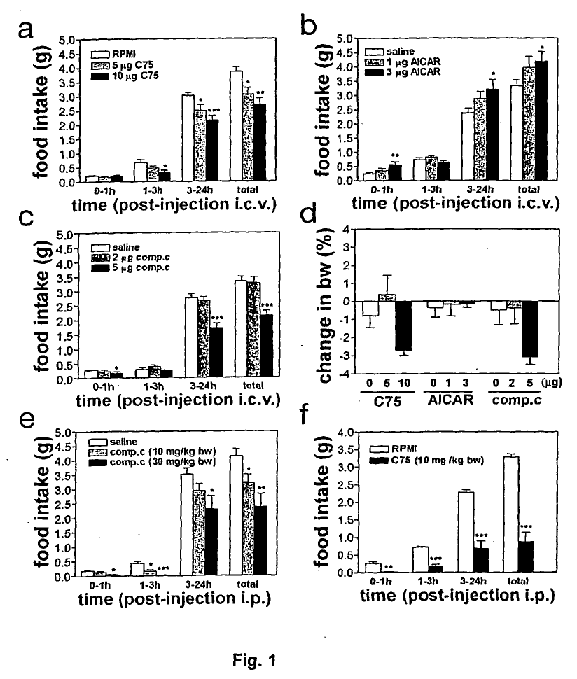

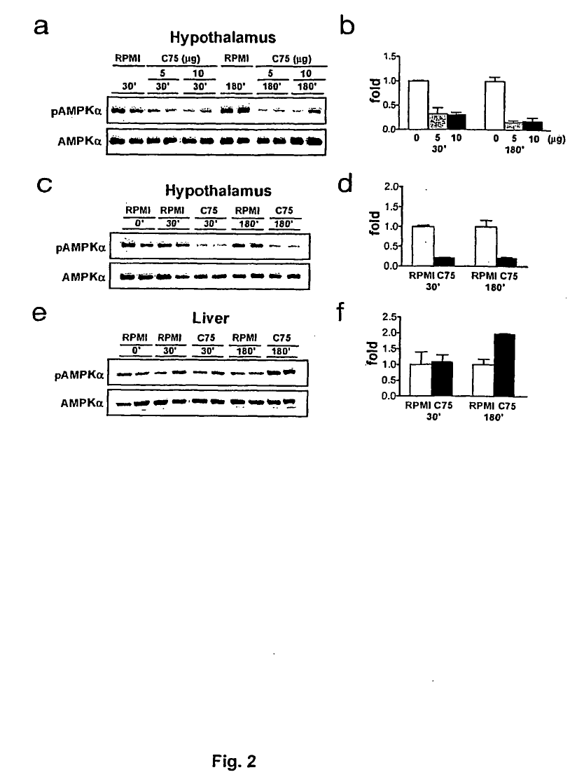

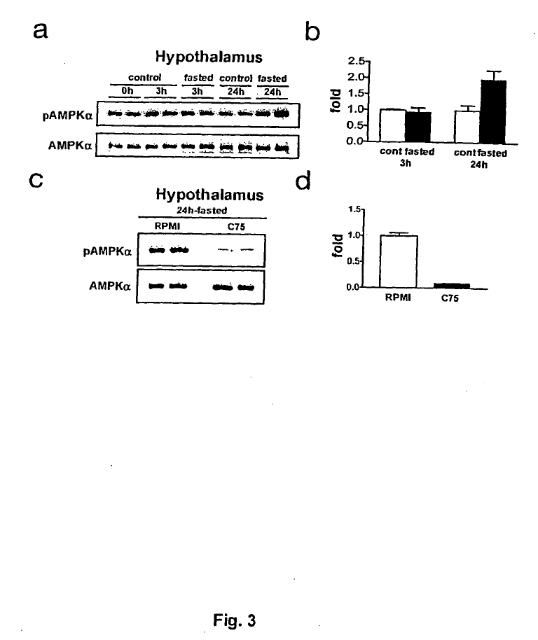

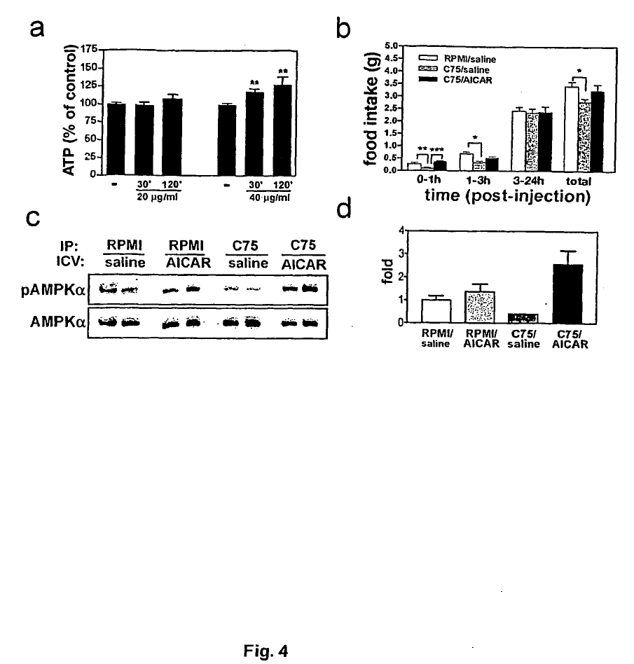

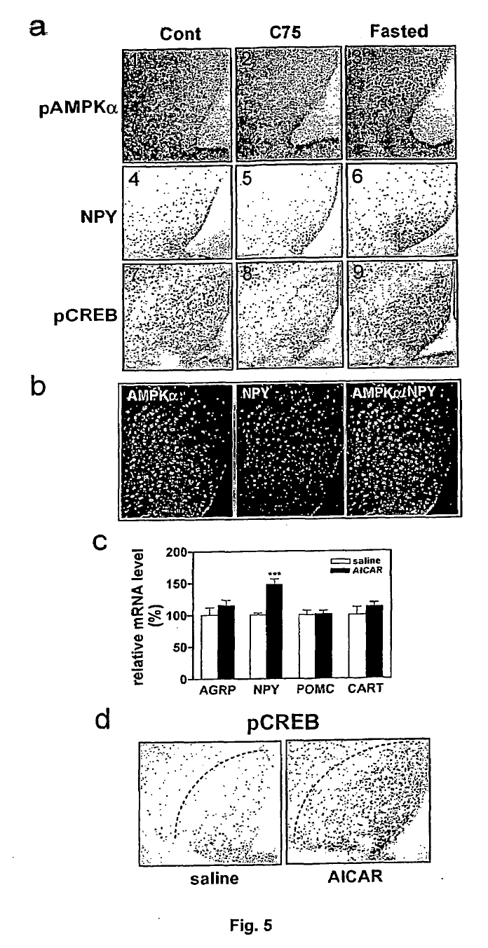

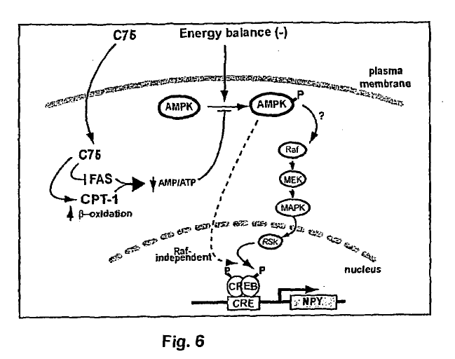

(b) Food intake was determined for mice (n=10-12) that received i.p. C75 (5 mg/kg bodyweight) followed by injection i.c.v. AICAR (3 mg) 1 hr later. Food intake was monitored at same time intervals as in FIG. 1a. *, p<0.05; **, p<0.01; ***, p<0.001 compared to RPMI/saline or C75/saline.

(c) Levels of hypothalamic phosphorylated and total of AMPKα were determined by Western blot in mice that received i.p. RPMI/i.c.v. saline, i.p. RPMI/i.c.v. AICAR, i.p. C75/i.c.v. saline or i.p. C75/i.c.v. AICAR using the same dosages as in FIG. 4b. (d) The graphs show the fold-difference from quantification of Western blot (FIG. 4c).

FIG. 5. C75 affects pAMPKα, NPY, and pCREB expression in the arcuate nucleus

(a) Immunohistochemistry of pAMPKα in situ hybridization of NPY (4, 5, 6) and immunohistochemistry of pCREB in the arcuate nucleus was performed using coronal brain sections from control, C75-treated (24 hr) and fasted (24 hr) mice.

(b) Colocalization of AMPKα2 (FITC) and NPY (Texas Red) in arcuate nucleus neurons by double fluorescent in situ hybridization.

(c) mRNA level of hypothalamic neuropeptides was determined by Northern analysis from mice (n=4 each) that received i.c.v. saline or AICAR (3 mg) 20 hr after injection. ***, p<0.001 compared to saline control.

(d) The arcuate pCREB levels (under dashed line) were shown from mice that received i.c.v. saline or AICAR (3 mg) 20 hr after injection. (e) A model for C75-induced changes in energy flux that alter AMPK activity to modulate CREB-NPY pathway signaling in the arcuate nucleus.

FIG. 6 shows a proposed mechanism by which changing the neuronal energy balance affects feeding.

### DETAILED DESCRIPTION OF THE INVENTION

As shown in FIG. 6, C75 and other compounds can affect feeding behavior. For example, certain compounds, when administered to a subject, can affect neuronal energy balance. Neuronal energy balance may be represented by the AMP/ATP ratio in the neuronal cells.

Thus, administration of a compound which increases ATP levels in hypothalmic neurons will decrease the neuronal energy balance, decreasing the subject's appetite. Determination of whether a compound will increase (or decrease) ATP levels in hypothalmic neurons is not difficult. One protocol is as follows: The neurons may be lysed on ice using TE buffer (100 mM Tris and 4 mM EDTA) and removed from the plate. ATP levels may then be measured in the linear range using the ATP Bioluminescence Kit CLS II (Roche, Indianapolis, Ind.) by following the manufacturer's protocol, with the results read by a Perkin-Elmer Victor² 1420.

Through its ability to inhibit FAS and stimulate CPT-1, C75 may increase ATP levels in hypothalamic neurons, as it does in the periphery and in cortical neurons. This change signals a positive energy balance, leading to a decrease in AMPK activity, resulting in a decrease in NPY expression. In fasting, when energy is depleted, AMPK is stimulated, thereby activating the CREB-NPY pathway and food intake. There appears to be relatively little change in the level of phosphorylated hypothalamic AMPK during normal feeding, and a prolonged period of decreased food intake is required before hypothalamic pAMPK levels increase. Hypothalamic AMPK appears responsive to changes in energy status due to C75 treatment or fasting. Thus, AMPK functions as a “fuel sensor” in the CNS.

The treatment of obesity remains a daunting medical problem. The present invention shows that one consequence of C75's actions is the alteration of AMPK activity. AMPK serves as a master fuel sensor, since C75's effects dominate over fasting-induced cues, and can even reduce food intake in ob/ob mice.

Compounds which either inhibit or stimulate AMPK may be used to regulate food intake. The compositions of the present invention can be presented for administration to humans and other animals in unit dosage forms, such as tablets, capsules, pills, powders, granules, sterile parenteral solutions or suspensions, oral solutions or suspensions, oil in water and water in oil emulsions containing suitable quantities of the compound, suppositories and in fluid suspensions or solutions. As used in this specification, the terms “pharmaceutical diluent” and “pharmaceutical carrier,” have the same meaning. For oral administration, either solid or fluid unit dosage forms can be prepared. For preparing solid compositions such as tablets, the compound can be mixed with conventional ingredients such as talc, magnesium stearate, dicalcium phosphate, magnesium aluminum silicate, calcium sulfate, starch, lactose, acacia, methylcellulose and functionally similar materials as pharmaceutical diluents or carriers. Capsules are prepared by mixing the compound with an inert pharmaceutical diluent and filling the mixture into a hard gelatin capsule of appropriate size. Soft gelatin capsules are prepared by machine encapsulation of a slurry of the compound with an acceptable vegetable oil, light liquid petrolatum or other inert oil.

Fluid unit dosage forms or oral administration such as syrups, elixirs, and suspensions can be prepared. The forms can be dissolved in an aqueous vehicle together with sugar, aromatic flavoring agents and preservatives to form a syrup. Suspensions can be prepared with an aqueous vehicle with the aid of a suspending agent such as acacia, tragacanth, methylcellulose and the like.

For parenteral administration fluid unit dosage forms can be prepared utilizing the compound and a sterile vehicle. In preparing solutions the compound can be dissolved in water for injection and filter sterilized before filling into a suitable vial or ampoule and sealing. Adjuvants such as a local anesthetic, preservative and buffering agents can be dissolved in the vehicle. The composition can be frozen after filling into a vial and the water removed under vacuum. The lyophilized powder can then be scaled in the vial and reconstituted prior to use.

Dose and duration of therapy will depend on a variety of factors, including (1) the subject's age, body weight, and organ function (e.g., liver and kidney function); (2) the nature and extent of the disease process to be treated, as well as any existing significant co-morbidity and concomitant medications being taken, and (3) drug-related parameters such as the route of administration, the frequency and duration of dosing necessary to effect a cure, and the therapeutic index of the drug. In general, does will be chosen to achieve serum levels of 1 ng/ml to 100 ng/ml with the goal of attaining effective concentrations at the target site of approximately 1 μg/ml to 10 μg/ml.

The following examples further elucidate, without limiting, the claimed invention.

### Methods

### Animals

All animal experiment was done in accordance with guidelines on animal care and use established by the Johns Hopkins University School of Medicine Institutional Animal Care and Use Committee.

Male BALB/c mice (7-9 weeks) were purchased from Charles River Laboratories (and housed in a controlled-light (12 hr light/12 hr dark cycle) environment (lights on 0200-1400 h) and allowed ad libitum access to standard laboratory chow and water. For fasting, food was withdrawn from cage at the onset of the dark cycle for 24 hr, but ad libitum access to water was allowed.

### Measurement of Food Intake.

Mice were implanted with permanent stainless steel cannulae into the lateral ventricle of the brain 0.6 mm caudal to Bregma, 1.2 mm lateral to the midline, and sunk to a depth of 2.2 mm below the surface of the skull. Implanted mice were housed in individual cages and utilized for i.c.v. and i.p. injections as indicated. C75 dissolved in RPMI1640 (Gibco-BRL), AICAR (Toronto Research Chemicals Inc) or compound C (46) (FASgen, Inc.) in saline was injected i.c.v., such that desired dose could be administered in a volume of 2.5 μl, while control groups received vehicle only. Injections were done immediately preceding dark onset and food intake measurements were taken at 1 hr (0-1 hr interval), 3 hr (1-3 hr interval), and 24 hr (3-24 hr interval) after dark onset. C75 i.p./AICAR i.c.v. treatment groups were i.p. injected with 5 mg/kg bodyweight C75 dissolved in 200 ml of glucose-free RPMI 1 hr before the dark onset, followed by 3 μg/2.5 μl saline i.c.v. AICAR immediately preceding the dark onset. Control groups received 200 μl of glucose free RPMI 1 hr before lights off and 2.5 μl of saline. Administration of i.p. compound C (10 or 30 mg/kg bodyweight) or C75 (10 mg/kg bodyweight) was followed by food intake measurement at the same times indicated.

### Western Blot Analysis

Hypothalami were dissected using as landmarks the optic chiasm rostrally, and the mammillary bodies caudally to a depth of 2 mm. Dissected hypothalamic and liver tissue were immediately frozen in liquid nitrogen. Tissues were homogenized in 200 μl of lysis buffer (50 mM Tris-HCl, pH 7.5, 250 mM sucrose, 5 mM sodium pyrophosphate, 50 mM NaF, 1 mM EDTA, 1 mM EGTA, 1 mM DTT, 0.5 mM PMSF, 0.1 mM benzamidine, 50 μg/ml leupeptin, 50 μg/ml soybean trypsin inhibitor). SDS detergent was added to a final 0.2%, and lysates were boiled for 5 min. After the supernatant was harvested, protein concentration was determined by BCA kit (Bio Rad). Phosphorylation of AMPKα was determined on a 4-15% gradient SDS-polyacrylamide gel using anti-phospho-AMPKα (α1 and α2, Thr172) antibody (1:1000, Cell Signaling). Anti-AMPKαt antibody (α1 and α2, 1:1000, Cell Signaling) was used as a loading control.

### Primary Hypothalamic Neuron Cultures and ATP Measurement

Hypothalami were dissected from E17 Sprague-Dawley rats (Harlan), and dissociated by trypsin (0.125%)-DNA (0.001%) solution and trituration as described by Landree, et al., J. Biol. Chem., 279, 3817-3827. Cells were plated at 6×10⁴ on poly-D-lysine coated 96 well plates (Corning Inc.) in neurobasal medium supplemented with B27, 0.5 mM L-glutamine, 1% penicillin-streptomycin (Gibco-BRL). To limit nonneuronal cell proliferation, cells were treated with cytosine arabinoside furanoside (1 μM) on day 4 after plating and 6-8 days-old cells were assayed for ATP. Hypothalamic neurons were lysed in TE (100 mM Tris-HCl, pH 7.4, 4 mM DTA), and ATP levels were measured within the linear range using the ATP BioLuminescence Kit CLSII (Roche) by following the manufacture's recommendation. Data were analyzed by a Perkin-Elmer Victor² 1420.

### RNA Preparation and Northern Blot Analysis.

Hypothalamic total RNA was purified using Trizol reagent (Gibco-BRL) and Northern blot analysis using 15 μg of total RNA was performed as described by Kim, et al, Am J Physiol Endocrinol Metab., 283, E867-879 (2002). RNA was hybridized with random primed ³²P-labeled DNA probes made from cloned plasmids of mouse AGRP (Genebank #U89486), human NPY (XM004941), rat CART (U10071), and mouse POMC (AH005319). As a loading control, the probe for mouse GAPDH gene was used at the same blot. The signals were quantified using an image analyzer (Molecular Dynamics) and Imagequant software.

### Immunohistochemistry.

Floating brain sections were prepared as described by Kim, et al, Am J Physiol Endocrinol Metab., 283, E867-879 (2002) with the modifications set forth by Shimuzu-Albergine, et al., J Neurosci 21, 1238-1246 (2001). Free-floating sections were blocked in PBS containing 5% goat serum, 0.1% BSA, 0.05% Triton-X100, 1 mM NaF for 1 hr at room temperature and incubated with anti-phospho-AMPKα (α1 and α2) antibody (1:100) or anti-phospho-CREB antibody (1:500, Cell Signaling) in PBS containing 1% goat serum, 0.1% BSA, 0.05% Triton-X100, 1 mM NaF overnight at 4° C. Signal was visualized by Vectastain ABC kit (Vector).

### In Situ Hybridization.

Anti-sense DIG-labeled NPY riboprobe was generated from a plasmid containing the NPY gene (XM004941). Hybridization and washing were performed as described by Kim, et al, Am J Physiol Endocrinol Metab., 283, E867-879 (2002). For double fluorescent in situ hybridization, DIG-labeled riboprobe was generated from plasmid containing AMPKα2 gene (pEBGα2, a gift from L. A. Witters) for AMPKα2 (FITC) and biotin-labeled riboprobe was used for NPY (Texas Red). Sheep FITC-conjugated anti-DIG antibody (1:50, Roche) was incubated in TNB buffer (100 mM Tris-HCl pH 7.5, 150 mM NaCl, and 0.5% blocking reagent) for FITC detection. Streptavidin-Texas Red (1:50, Amersham Pharmacia), rabbit anti-Texas Red antibody (1:50, Molecular Probes), goat biotin-conjugated anti-rabbit IgG antibody (1:50, Santa Cruz Biotechnology) and streptavidin-Texas Red (1:30) were incubated serially in TNB buffer for Texas Red detection.

### Analysis and Quantification of Images.

Images of in situ hybridization and immunohistochemistry were visualized using an Axiocam HRc digital camera (Carl Zeiss) and images were acquired using Improvision Openlab software, and quantified by NIH Image program (Macro).

### Statistical Analysis

Data are presented as means ± standard error of the mean from multiple determinations (n>4). Unless otherwise noted, data were analyzed by One-way ANOVA with Dunnett post test to compare treated samples with controls. Differences from post tests were considered statistically significant at *, P<0.05; **, P<0.01; ***, P<0.001. For the analysis of AMPK activity results (FIGS. 5C and D) each time point was compared with control samples by performing unpaired one-tailed t-tests.

### Primary Cortical Neuronal Cultures

Cortices were removed from E17 Sprague-Dawley rats (Harlan, Indianapolis, Ind.), and were dissociated by mild trypsinization and trituration as described by Dawson, et al. J Neurosci 13, 2651-2661 (1993). Cells were plated on poly-D-lysine coated plastic Nunclon culture dishes at a density of 5×10⁵ cells/cm² in Minimum Essential Media (MEM) supplemented with horse serum, fetal bovine serum, glutamine, and the antibiotics gentamycin and kanamycin. Cells were plated onto vessels as required for each type of experiment: T-25 flasks for oxidation assays; 6-well plates for Western blots, SAMS peptide assays, and HPLC analysis; 24 well plates for FAS and CPT-1 activity assays; 4 well chamber slides for immunocytochemistry; and 96 well plates for the determination of ATP levels and cell viability assays. For standard cultures cells were treated with cytosine arabinoside on day 4, and were assayed after 7-10 days in vitro. For cultures overgrown with glia, cells were not treated with cytosine arabinoside and were used for immunocytochemistry on day 6. Drug treatments were performed with vehicle or C75, resuspended in RPMI; cerulenin (Sigma) resuspended in RPMI; and 5-(tetradecyloxy)-2 Furoic Acid (TOFA) resuspended in 100% DMSO.

### Immunocytochemistry

Cortical neurons were grown as described and harvested 7 days after plating for immunocytochemistry. Cells were fixed with 4% PFA and 20% sucrose for 20 min at 4° C., and permeated with 0.2% Triton X-100 in PBS for 10 min at 4° C. As these cultures normally contain less than 1% glial cells, cultures were also prepared in which glia were allowed to overgrow, as described, to better evaluate the expression of FAS and AMPK in glia. Cells were incubated in blocking solution (PBS containing 4% normal serum) for 1 hr at 4° C. Primary antibodies against the following antigens were diluted in blocking solution overnight at 4° C.: glia fibrillary acidic protein (GFAP) (Chemicon International Temecula, Calif.) 1:1000; neuron-specific tubulin (NST) (Bacbo, Richmond Va.) 1:1000; AMPKα (1:500); and FAS (1:1000). Cells were incubated for 1 hr at room temperature with secondary antibodies conjugated with FITC for NST and GFAP staining, or with rhodamine for FAS and AMPK staining.

### Measurement of Acetate Incorporation

Cells were pre-treated with the indicated concentrations of vehicle or C75 for 15 min in conditioned media, and then labeled with 100 μM ³H Acetic Acid (NEN) for an additional 1.75 hours as previously described by Pizer, et al., Cancer Research 1996, 745-751 (1996). Lipids were extracted with chloroform/methanol, dried under N₂ and counted using a liquid scintillation counter.

### Measurement of ATP

Neurons were lysed on ice using TE buffer (100 mM Tris and 4 mM EDTA) and removed from the plate. ATP levels were then measured in the linear range using the ATP Bioluminescence Kit CLS II (Roche, Indianapolis, Ind.) by following the manufacturer's protocol, and results were read by a Perkin-Elmer Victor² 1420.

### Cell Viability Assay

Cortical neurons were treated for the indicated times with the indicated doses of drug, and viability was determined using the Live/Dead Viability/Cytotoxicity Kit (Molecular Probes, Eugene, Oreg.). The conversion of the cell permeant non-fluorescent calcein AM dye to the intensely fluorescent calcein dye is catalyzed by intracellular esterase activity in live cells and is measured by detecting the absorbance at 485 nm/535 nm using the Perkin-Elmer Victor² 1420.

### HPLC

Adenine nucleotide levels in primary cortical neuron lysates were determined by HPLC analysis as described by Stocchi, et al. Anal Biochem 167, 181-190 (1987). Briefly, each well of a 6 well plate was washed with 2 ml of ice cold PBS, and lysed with 70 μl of ice cold 0.5 M KOH and scraped. One hundred and forty μl of H₂0 were added to lysates and incubated on ice for 5 min, and the pH was then adjusted to 6.5 by addition of 1 M KH₂PO₄. Cell lysates were spun through Microcon YM-50 centrifugal filters and stored at −80° C. for subsequent HPLC analysis. The HPLC used was an Agilent 1100 LC with a variable wavelength detector. The analysis was done using Chemstation A.10.01 software.

### Measurement of Fatty Acid Oxidation

Fatty acid oxidation was measured as described by Watkins, et al., Arch Biochem Biophys, 289, 329-336 (1991). Briefly, primary cortical neurons adherent to the flask were treated in triplicate with C75 at the indicated doses for the indicated times in of HAM-F10 media supplemented with 10% FBS. One-half μCi/ml (20 nmol) of [1¹⁴C]-palmitic acid (Moravek Biochemicals, Brea, Calif.) resuspended in α-cyclodextran (10 mg/ml in 10 mM Tris) and 2 μM carnitine was added for the last 30 min of each treatment. Flasks were fitted with serum stoppers and plastic center wells (Kontes, Vineland, N.J.) containing glass microfiber filters (presoaked in 10 μl of 20% KOH). Following the incubation, 200 μl of 2.6 N HClO₄ was injected into the flasks and the ¹⁴CO₂ was trapped for 2 hr at 37° C. The filters were removed and quantified by liquid scintillation counting. The contents of the flasks were then hydrolyzed with 200 μl of 4 N KOH and neutralized using H₂SO₄. The water soluble products were extracted using CHCl₃/MeOH and H₂O and quantified by liquid scintillation counting. The total amount of fatty acid oxidation was obtained by addition of the ¹⁴CO₂ and water soluble products and represented as % of control or as a specific activity (nmol/hr/mg).

### Measurement of Glucose Oxidation

Glucose oxidation assays were based on the work described by Rubi, et al., Biochem J 364, 219-226 (2002). Neurons adherent to the flask were treated in triplicate with C75 at the indicated doses for the indicated times in Krebs-Ringer bicarbonate HEPES buffer (KRBH buffer: 135 mM NaCl, 3.6 mM KCl, 0.5 mM NaH₂PO₄, 0.5 mM MgCl₂, 1.5 mM CaCl₂, 5 mM NaHO₃ and 10 mM HEPES) containing 1% BSA and 10 mM D-glucose. One-half μCi/ml [U-¹⁴C]-glucose (NEN) was added for the last 30 min of each treatment and flasks were fitted as described for fatty acid oxidation assays. Reactions were stopped with the injection of 7% perchloric acid into the flask, and then 400 μl of benzethonium hydroxide was injected into the center well. After 2 hr at 37° C., complete oxidation was quantified by measuring the amount of ¹⁴CO₂ in the center well by liquid scintillation counting, and represented as % of control or as a specific activity (pmol/hr/mg).

### Measurement of CPT-1 Activity

CPT-1 activity was measured using digitonin permeabilization as described by Sleboda, et al., Biochimica et Biophysica Acta, 1436, 541-549 (1999). Drugs and vehicle controls were added as indicated for each experiment. After 2 hr, the medium was removed, cells were washed with PBS, and incubated with 700 μl of assay medium consisting of: 50 mM imidazole, 70 mM KCl, 80 mM sucrose, 1 mM EGTA, 2 mM MgCl₂, 1 mM DTT, 1 mM KCN, 1 mM ATP, 0.1% fatty acid free bovine serum albumin, 70 μM palmitoyl-CoA, 0.25 μCi [methyl-¹⁴C]L-carnitine (Amersham Pharmacia Biotech, Piscataway, N.J.), 40 μg digitonin, with or without 100 μM malonyl-CoA. After incubation for 6 min at 37° C., the reaction was stopped by the addition of 500 μl of ice-cold 4 M perchloric acid. Cells were then harvested and centrifuged at 13,000×g for 5 min. The pellet was washed with 500 μl ice-cold 2 mM perchloric acid and centrifuged again. The resulting pellet was resuspended in 800 μl dH₂O and extracted with 400 μl of butanol. The butanol phase, representing the acylcarnitine derivative, was measured by liquid scintillation counting.

### Measurement of AMP-Activated Protein Kinase Activity

AMPK activity was determined by performing SAMS peptide assays as described by Witters, et al., J Biol Chem 267, 2864-2867 (1992). Neurons plated on 6 well culture dishes were lysed using 350 μl per well of Triton X-100 lysis buffer: 20 mM Tris-HCl, pH 7.4, 50 mM NaCl, 1% Triton X-100, 250 mM sucrose, 50 mM NaF, 5 mM NaPPi, 1 mM dithiothreitol, 50 μg/ml Leupeptin, 0.1 mM Benzamidine, and 50 μg/ml trypsin inhibitor. Three wells were pooled per condition, and AMPKα was immunoprecipitated in the presence anti-AMPKα (2-20) antibody coupled to Protein A/G beads (Santa Cruz, Calif.). Immunoprecipitates were then washed and resuspended in 4× assay buffer and kinase activity was assessed by measurement (for 20 min at 30° C.) of the incorporation of ³²P into the synthetic SAMS peptide substrate, HMRSAMSGLHLVKRR, (Princeton Biomolecules). Samples were spotted on P81 phosphocellulose paper, washed extensively, and quantitated by Cerenkov counting. Each sample was corrected for protein concentration and reported either as % of control or as pmol/min/mg.

### Electrophysiology and mEPSC Analysis

Whole cell patch clamp recordings were performed from cortical cultures at 14-21 days in vitro. To isolate AMPA-mediated mEPSCs, neurons were continuously perfused with artificial cerebral spinal fluid (aCSF) at a flow rate of <1 ml/min. The composition of aCSF was as follows (in mM): 150 NaCl, 3.1 KCl, 2 CaCl₂, 2 MgCl₂, 10 HEPES, 0.1 DL-APV, 0.005 strychnine, 0.1 picrotoxin, and 0.001 tetrodotoxin (TTX). The osmolarity of the aCSF was adjusted to 305-310, pH was 7.3-7.4. Intracellular saline consisted of (in mM): 135 CsMeSO₄, 10 CsCl, 10 HEPES, 5 EGTA, 2 MgCl₂, 4 Na-ATP, and 0.1 Na-GTP. This saline was adjusted to 290-295 mOsm, pH 7.2.

Once the whole-cell recording configuration was achieved, neurons were voltage clamped and passive properties were monitored throughout. In the event of a change in Rs or Ri greater than 15% during the course of a recording the data were excluded from the set. mEPSCs were acquired through an Axopatch 200B amplifier (Axon Instruments, Union City, Calif.), filtered at 2 kHz and digitized at 5 kHz. Sweeps (20 seconds) with zero latency were acquired until a sufficient number of events were recorded (minimum of 5 minutes). Data was continuously recorded only after a period of 1-2 minutes where the cell was allowed to stabilize. mEPSCs were manually detected with MiniAnalysis (Synaptosoft Inc, Decatur, Ga.) by setting the amplitude threshold to √RMS*3 (usually 4 pA). Once a minimum of 100 events was collected from a neuron, the amplitude, frequency, rise time (time to peak), decay time (10%-90%), and passive properties were measured. In all electrophysiological experiments, a similar amount of data (n) was acquired from each experimental group (i.e. DMSO, Drug). Data from each group was then averaged and statistical significance determined by the student T test. Data were never reused or transferred from one experimental group to another (DMSO controls were exclusive).

### EXAMPLE 1

### Feeding Behavior is Changed by C75, AICAR or Compound C Treatment

Mice were implanted with intracerebroventricular (i.c.v.) cannulae to measure food intake after dark onset administration of C75 (FIG. 1a). All mice had access to food ad libitum during the 24 hr cycle. C75 significantly reduced food intake during the 1-3 and 3-24 hr time intervals in a dose-dependent manner (FIG. 1a). Injection of 5 and 10 mg of C75 caused a 20.3% (p<0.05) and 37.7% (p<0.01) reduction in food intake over 24 hr, respectively. The 10 μg dose also produced a reduction in body weight (FIG. 1d). These results indicate that C75 reduces food intake via a central mechanism.

AICAR (5-aminoimidazole-4-carboxamide-1-b-D-ribofuranoside), a compound that stimulates AMPK activity, is taken up into cells and phosphorylated to form ZMP (see, Sabina, et al., J Biol Chem, 260, 6107-14 (1985)), which mimics the effects of AMP on AMPK activation (see, Sullivan, J. E., et al., FEBS Lett 353, 33-6 (1994)). In contrast to the feeding inhibition produced by C75, i.c.v., administration of AICAR increased food intake. A dose of 3 μg increased food intake to 230% (p<0.01) within 1 hr, 135% (p<0.01) at 3-24 hr and total 24 hr food intake was increased to 130% of control (p<0.05) (FIG. 1b). Despite this increase in food intake, this single dose of AICAR has no significant effect on bodyweight (FIG. 1d). As reported by Abu-Elheiga, L., et al. (Science, 291, 2613-6 (2001)), bodyweight does not always change in proportion to food intake. A previous report noted that chronic subcutaneous injection of AICAR (1 g/kg bodyweight) for 4 weeks had no impact on either food intake or bodyweight (Winder, W. W., et al., J Appl Physiol 88, 2219-26 ((2000))), but that there was a reduction in fat pad mass and an increase in liver mass. Thus, i.c.v. administration of a single dose of AICAR may have an effect on the mass of these peripheral tissues, such that bodyweight does not change despite increased food intake.

To confirm the effect of AMPK on food intake, we used compound C, which is a selective AMPK inhibitor (Zhou, G., et al., J Clin Invest 108, 1167-74 (2001).). The i.c.v. injection of 5 mg compound C caused a 36.2%, 37.8% and 35.6% reduction in food intake at 0-1, 3-24 hr and over 24 hr, respectively (FIG. 1c). This dosage of compound C led to a weight loss (FIG. 1d). Interestingly, as with the stimulatory effect of AICAR on feeding, the inhibitory effect of compound C on feeding was profound at 0-1 hr and 3-24 hr. The intraperitoneal (i.p.) injection of compound C also had a similar reduction in food intake (FIG. 1e), showing that a higher dose (30 mg/kg bodyweight) decreased food intake during all time intervals (27.4%, 3.68%, 65.7% and 57.8% of control during 0-1, 1-3, 3-24 hr and total, respectively). Even though AICAR or compound C may have additional cellular effects that cannot be excluded, the opposite results on food intake obtained using an AMPK activator and inhibitor shows that AMPK is involved in feeding behavior. We determined the time course of action of the i.p. C75 administration, with the intention of utilizing this route of administration for C75 in further experiments designed to compare the central and peripheral effects of C75 on the change in AMPK activation, and to combine C75 and AICAR treatments. Administration of i.p. C75 (10 mg/kg bodyweight) caused a dramatic decrease in food intake during all intervals measured (8.3%, 23.3%, and 30.1% of control during 0-1, 1-3, and 3-24 hr, respectively) (FIG. 1f). Total 24 hr-food consumption was significantly reduced to 26.3% of control (p<0.001). The effect of C75 on food intake was more pronounced and lasted longer than that of compound C. The greater magnitude of the effect following peripheral administration of C75 on food intake may reflect the larger dose that can be administered via this route, or an additional peripheral effect, compared to the i.c.v. route of administration.

Collectively, these results demonstrate that C75 and compound C (administered either i.c.v. or i.p.) produce opposite effects on food intake over similar time courses compared to i.c.v. administration of AICAR.

### EXAMPLE 2

### C75 Decreases the Phosphorylation of Hypothalamic AMPK

The hypothalamus plays an important role in monitoring energy balance and integrating peripheral signals that affect food intake. Although the expression of AMPK in brain has been reported, its function in the brain was previously unknown. C75 inhibits FAS and stimulates carnitine palmitoyl transferase-1 (CPT-1), the enzyme that imports palmitate into the mitochondrion for β-oxidation. Both of these actions may signal a positive energy balance in neurons of the hypothalamus, which may inactivate hypothalamic AMPK. To examine the effect of C75 on hypothalamic AMPK activity, we determined the effect of C75 treatment on the level of phosphorylation of the α catalytic subunit of AMPK (pAMPKα) in the hypothalamus, which correlates with its activity (FIG. 2).

Mice received vehicle, 5 μg, or 10 mg of C75 i.c.v., and the levels of hypothalamic pAMPKα a were determined by Western blot. The level of AMPKα (α1 and α2 subunits) served as a loading control. Compared to levels of pAMPKα in vehicle-treated control animals, C75 reduced the levels of pAMPKα (α1 and α2) in the hypothalamus at 30 min and 3 hr three- and six-fold, respectively (FIGS. 2a,b). As seen with central administration of C75, i.p. injection of C75 (10 mg/kg body weight) significantly reduced the levels of pAMPKα in the hypothalamus at 30 min and 3 hr (FIGS. 2c,d). In contrast, C75 had little effect on pAMPKα levels in the liver 30 min after administration, but increased pAMPKα levels at 3 hr (FIGS. 2e,f). These results demonstrated that C75 rapidly decreased AMPK activity in the hypothalamus. The decrease in hypothalamic pAMPKα levels could result from the metabolic changes that occur as a result of FAS inhibition, which would diminish energy expenditure and signal a favorable energy balance. These results also indicate that the phosphorylation of AMPK is regulated differently in the hypothalamus than in the liver in response to C75. This difference most likely reflects differences between metabolic pathways, or the flux through those pathways, found in neurons and in liver. By 3 hr, the decreased food intake seen with C75 treatment may signal an energy poor state in liver (FIG. 2e), leading to AMPK activation, indicating an attempt to preserve energy levels through the stimulation of fatty acid oxidation, for example.

### C75 Decreases the Fasting-Induced Phosphorylation of Hypothalamic AMPK

It has been shown that the activity of AMPK is elevated in fasted rat liver. To investigate whether hypothalamic AMPK is responsive to fasting, the level of pAMPKα was monitored after withdrawal of food at the onset of dark cycle in mice fed ad libitum. There was no change in pAMPKα levels within 3 hr of food withdrawal (FIGS. 3a,b). However, fasting for 24 hr resulted in a two-fold stimulation in the level of hypothalamic pAMPKα (FIGS. 3a,b). While Davies, et al. (FEBS Lett, 377, 421-5 (1995)). noted no difference in AMPK activity between dark and light cycles in rats fed ad libitum, only one time point (6 hr) was investigated, without correlation to feeding profile in the interval before this measurement was made. Our results show that the activation of hypothalamic AMPK could be involved in the fasting-induced stimulation of food intake.

We next examined whether C75 could reduce AMPK phosphorylation in the setting of fasting, when AMPK phosphorylation is increased. This is important in establishing a link between C75-induced FAS inhibition and AMPK activity, as C75 does inhibit feeding even in fasted mice. After 24 hr of fasting, either vehicle (RPMI) or C75 was administrated i.p., and the levels of hypothalamic pAMPKa were determined. C75 treatment profoundly reduced the level of pAMPKa compared to that of control (FIGS. 3c.d). Given our observation that C75 suppresses food intake even in fasted mice, the ability of C75 to reduce the levels of pAMPKα in fasted mice supports that C75 might inhibit feeding by an AMPK-mediated mechanism.

### C75 Increases the Hypothalamic Neuronal ATP Level

It has been shown that C75 increases ATP levels in 3T3-L1 adipocytes and even in primary cortical neurons. Since an increase in the AMP/ATP ratio is known to activate AMPK, we hypothesized that a C75-induced increase in hypothalamic ATP levels could contribute to a decrease in AMP/ATP, resulting in reduced hypothalamic AMPK activity. Treatment of primary cultures of hypothalamic neurons with 40 mg/ml C75 led to a significant increase in neuronal ATP levels to 118 and 128% of control at 30 min and 2 hr, respectively (FIG. 4a). C75 treatment caused a similar change in ATP levels in primary cortical neurons, producing a decrease in the ratio of AMP/ATP and inactivation of AMPK. Therefore, It is likely that an increase in ATP caused by C75 also contributed to the decrease in AMPK activity in the hypothalamus.

### AICAR Reverses C75's Anorexic Effect and Increases the Phosphorylation of Hypothalamic AMPK

To determine whether AICAR could reverse the C75-induced decrease in food intake, we treated mice 1 hr before the onset of dark cycle with either vehicle or C75 (5 mg/kg bodyweight) i.p., followed 1 hr later by an i.c.v. injection of vehicle or AICAR (3 mg) (FIG. 4b). C75 reduced food intake at 1 hr to 37.5% of control (RPMI/saline) (p<0.01). In contrast, AICAR treatment increased food intake at 1 hr to 346% of the amount of C75/saline treatment (p<0.001). AICAR treatment reversed the C75-induced anorexia, resulting in food intake that was similar to that of control vehicle-treated mice. The effect of AICAR on C75-treated mice was of limited duration, consistent with the metabolism of AICAR. The lack of an effect on food intake during the 3-24 hr time interval may represent the net effect of the opposing actions of C75 and AICAR. If the reversal of C75-mediated anorexia by AICAR involves alteration of AMPK activity, ICAR should similarly reverse the decrease in the level of hypothalamic pAMPKα that occurs with C75 treatment. Ad libitum fed mice received an i.p. injection followed by an i.c.v. injection 1 hr later as follows: i.p. RPMI and i.c.v. saline; i.p. RPMI and i.c.v. AICAR; i.p. C75 and i.c.v. saline; and i.p. C75 and i.c.v. AICAR (FIG. 4c). Hypothalamic tissues were prepared for Western blot 30 min after the i.c.v. injection (FIGS. 4c,d). A low level of pAMPKα was detected in vehicle-treated mice, which was increased in AICAR-treated animals (FIGS. 4c,d). Mice that received C75 i.p. and saline i.c.v. displayed a profound decrease in pAMPKα levels.

AICAR treatment following C75 treatment completely reversed the C75-induced decrease in hypothalamic pAMPKα levels. Sub-threshold doses would have been used with only behavioral data, but the fact that AICAR prevented the C75 induced changes in both behavior and the status of AMPK phosphorylation support a common site of action for the effects of C75 and AICAR. These results indicate that AICAR restores both C75-induced anorexia and the C75-induced suppression of AMPK activity.

### C75 Alters pAMPK, pCREB and NPY Expression in Arcuate Neurons.

AMPK acutely regulates cellular metabolism and chronically regulates gene expression. To ascertain whether the changes in the phosphorylation status of AMPK in the hypothalamus reflected the level of pAMPKα in the arcuate nucleus, we performed immunohistochemistry for pAMPKα using coronal brain sections containing the arcuate nucleus (FIG. 5a1-3). pAMPKα was detected in the arcuate nucleus of mice fed ad libitum (FIG. 5a1), and immunostaining was successfully blocked by preabsorbing with phospho-AMPKa peptide (data not shown). Compared to control, pAMPKa immunoreactivity was increased to 171% of control in the arcuate nucleus of mice fasted for 24 hr (FIG. 5a3). pAMPKa-immunoreactivity was reduced in C75-treated mice to 12% of control, even in the setting of reduced food intake (FIG. 5a2). These changes are consistent with our Western blot data (FIGS. 2a,c), and confirm that C75 reduces pAMPKα levels in the arcuate nucleus.

We have previously demonstrated by Northern blot analysis that C75 decreased hypothalamic NPY expression (4, 9). We next investigated whether the decreases in pAMPKa in the arcuate correlated with changes in NPY that occur with C75 treatment. NPY expression in neurons within the arcuate nucleus was determined in control, C75-treated, and fasted mice (FIG. 5a4-6). Consistent with previous Northern blot analysis of hypothalamic tissues (9), NPY mRNA expression was down regulated in the arcuate nucleus of C75-treated mice to 66% of control (FIG. 5a5) and up regulated in fasted mice to 140% of control (FIG. 5a6). It has been shown that the cAMP-CREB pathway mediates NPY expression under fasted conditions (37, 38), suggesting that leptin modulates NPY gene expression through this pathway (38). To elucidate the pathways involved in the down-regulation of NPY that occurs with C75 treatment, we determined the level of pCREB in the arcuate nucleus (FIG. 5a7-9). As previously reported (38), 24 hr fasting increased pCREB immunoreactivity in the arcuate nucleus to 197% of control (FIG. 5a9). In contrast, C75 decreased the level of pCREB to 39% of control (FIG. 5a8), consistent with the hypothesis that the decrease in NPY gene expression caused by C75 may be mediated by a decreased level of pCREB. To clarify the co-localization of AMPK and NPY in the arcuate nucleus, double in situ hybridization was performed (FIG. 5b). A subpopulation of neurons in the arcuate nucleus that expressed AMPKa2 mRNA also expressed NPY mRNA (FIG. 5b). It is known that NPY and CREB co-localize to neurons in the arcuate nucleus. These results indicate that AMPK, NPY, and CREB are co-expressed in a subpopulation of neurons within the arcuate nucleus, and support the hypothesis that AMPK may modulate CREB phosphorylation to affect NPY expression.

In contrast to C75, AICAR had the opposite effect (FIGS. 5c,d). Thus, consistent with our findings that AICAR stimulated feeding, AICAR significantly increased hypothalamic NPY expression 20 hrs after i.c.v. administration (FIG. 5c). The increase in NPY expression seen with AICAR treatment may mediate the stimulation of food intake seen at later times (3-24 hr) in FIG. 1b. Since no change in NPY expression with AICAR treatment was detected within 5 hr (data not shown), it appears that the earlier change in feeding (0-1 hr) is mediated by NPY gene expression-independent mechanism. AICAR also increased pCREB level in the arcuate up to 231% of control (FIG. 5d), which supports that AMPK may modulate CREB phosphorylation.

1. A method for regulating food intake by administering a compound to a subject, wherein the compound affects the activity of AMPK.

2. The method of claim 1, wherein the AMPK is hypothalmic AMPK.

3. The method of claim 1, wherein the compound is not C75.

4. The method of claim 1, wherein the compound is C75.

5. The method of claim 1, wherein the compound inhibits AMPK

6. The method of claim 1, wherein the compound stimulates AMPK.

7. The method of claim 1, wherein the subject is a human.

8. The method of claim 1, wherein the subject is a mammal but is not a human.

9. A method of altering food intake in a subject by administering a compound to the subject which changes the subject's neuronal energy balance.

10. The method of claim 9, wherein the compound decreases the subject's neuronal energy balance.

11. The method of claim 9, wherein the compound increases the subject's neuronal energy balance.

12. The method of claim 9, wherein the subject is a human.

13. The method of claim 9, wherein the subject is a mammal but is not a human.

14. The method of claim 9, wherein the compound is not C75.
